(19) United States
(12) Patent Application Publication
(10) Pub. No.: US 2002/0035081 A1
(43) Pub. Date: Mar. 21, 2002

(54) Inhibition of hepatitis B replication

(76) Inventors: Jack R. Wands, Waban, MA (US); Pier Paolo Scaglioni, Boston, MA (US); Margherita Melegari, Boston, MA (US)

Correspondence Address: JANIS K. FRASER, Fish & Richardson P.C., 225 Franklin Street, Boston, MA, 02110-2804, US

(21) Appl. No.: 09/812,862

(22) Filed: Mar. 20, 2001

Related U.S. Application Data
(62) Division of application No. 09/372,548, filed on Aug. 11, 1999, now abandoned.
Application No. 09/372,548 is a continuation of application No. 08/667,073, filed on Jun. 20, 1996, now abandoned.
(60) Provisional application No. 60/017,814, filed on Jun. 20, 1995.

(51) Int. Cl. A61K 48/00; C07H 21/04; C12N 15/869

(52) U.S. Cl. 514/44; 424/93.21; 536/23.72; 435/456

(57) ABSTRACT

The invention features a polypeptide having a first amino acid sequence of at least 70 amino acids in length that is identical to a region of a wild type HBV core protein; and lacks a second amino acid sequence of the wild type HBV core protein, where the second sequence includes the carboxyterminal three amino acids of the wild type HBV core protein and does not exceed nine amino acids in length.

### CROSS-REFERENCE TO RELATED APPLICATIONS

This application is a divisional of U.S. patent application Ser. No. 09/372,548, filed Aug. 11, 1999, now pending, which is a continuation of U.S. patent application Ser. No. 08/667,073, filed Jun. 20, 1996, now abandoned, which claims priority from U.S. Provisional Patent Application Ser. No. 60/017,814, filed Jun. 20, 1995.

### STATEMENT AS TO FEDERALLY SPONSORED RESEARCH

The invention was funded in part by grants CA-35711 and AA-08169 from the National Institutes of Health. The Government has certain rights to this invention.

### BACKGROUND OF THE INVENTION

The invention relates to treating infections of a hepadnavirus, e.g., hepatitis B virus.

Hepatitis B virus (HBV) is a member of the hepadnavirus family, a group of enveloped DNA viruses that cause acute and chronic hepatitis. Major clinical consequences of HBV infection include acute liver failure, liver cirrhosis, and primary hepatocellular carcinoma (HCC). With more than 250 million individuals infected worldwide, effective treatment of chronic HBV infection is a major public health goal (Ganem et al., Annu. Rev. Biochem., 56:651-693, 1987). Although an effective and inexpensive vaccine is available for preventing infection, to date there is no effective therapy for treating individuals with persistent infection, nor for reducing the risk of liver disease in infected patients (Maynard et al., Rev. Infect. Dis., 11,S574-S578, 1989); DiBisceglie et al., Cancer Detection and Prevention, 14, 291-293, 1989). Current treatments for chronic HBV infection include interferon and other inhibitors of viral DNA synthesis. Since these agents have achieved only limited success, additional antiviral approaches are urgently needed.

Hepadnaviruses are composed of a viral envelope, a nucleocapsid which contains a relaxed circular 3.2 kb DNA genome, and a virally encoded reverse transcriptase. Following infection of a cell, virion DNA is delivered to the nucleus where it is converted into a covalently closed circular DNA (cccDNA), which is in turn transcribed into several subgenomic and pregenomic mRNAs. The pregenomic RNA is then encapsulated into the viral nucleocapsid, together with the reverse transcriptase enzyme necessary to generate the viral DNA genome (Enders et al., J. Virol., 67, 35-41, 1987). Selective encapsulation of pregenomic RNA depends on both nucleocapsid protein and on viral polymerase (Bartenschlager et al., J. Virol., 64, 5324-5332, 1990; Hirsch et al., Nature, 344, 552-555, 1990; Nassal, M., J. Virol., 66, 4107-4116, 1992; Roychoury et al., J. Virol., 65, 3617-3624, 1991) as well as on a cis-acting encapsulation signal located at the 5′ end of the pregenomic RNA (Bartenschlager et al., supra; Junker-Niepmann et al., EMBO J., 9, 3389-3396, 1990; Pollack et al., J. Virol., 67, 3254-3263, 1993.

The mammalian hepadnavirus 21 kd core protein is a 183-187 (depending on the viral strain) amino acid monomer, 180 of which self assemble into an icosahedral structure within the cytoplasm of infected cells. The core protein has two functional domains. The aminoterminus (amino acids 1 to 139-44) is essential for core assembly. A carboxyterminal arginine-rich region (amino acids 139-183, or 144-187, depending upon the viral strain) binds nucleic acids that are required for positive strand DNA synthesis, and stabilizes core particles for complete assembly of the complex into an enveloped viral particle (Birnbaum et al. J. Virol., 64, 3319-3330, 1990; Yu et al., J. Virol., 65,2511-2517, 1990; Nassal, M., supra).

### SUMMARY OF THE INVENTION

The invention is based on Applicants' discovery that altering the carboxyterminus of the hepadnavirus core protein creates a mutant polypeptide that reduces replication of a wild type hepadnavirus, by a dominant negative mechanism. The inhibitory effect is achieved by deletion of a few carboxyterminal amino acids from the core protein, and/or by joining the core protein to a hepadnavirus surface protein, thereby creating a core-surface fusion polypeptide.

Accordingly, the invention features a method of inhibiting the replication of a naturally-occurring, infectious hepadnavirus. The method involves introducing into the proximity of the hepadnavirus a hepadnavirus mutant polypeptide, or a nucleic acid that encodes such a hepadnavirus mutant polypeptide. The polypeptide includes a first amino acid sequence that is substantially identical to a region of a wild type hepadnavirus core protein, but lacks a second amino acid sequence of the wild type hepadnavirus core protein, wherein the second sequence includes the carboxyterminal three amino acids of the wild-type hepadnavirus core protein and does not exceed 100 amino acids in length. The mutant polypeptide is introduced into the infected cell, or is expressed from the nucleic acid, in the proximity of the naturally-occurring hepadnavirus, so as to be available to inhibit replication of the hepadnavirus.

When the method of inhibiting hepadnavirus replication is targeted against HBV, the carboxyterminal amino acid of the first amino acid sequence can be selected from the group consisting of any of the amino acids between position 81 and position 180 of the sequence shown in FIG. 7 (SEQ ID NO: 12), inclusive; preferably the carboxyterminal amino acid is chosen from the group consisting of the amino acids between position 171 and position 180 of the sequence shown in FIG. 7 (SEQ ID NO: 12), inclusive. A construct exemplified herein ends with a carboxyterminal residue at position 171, so that the mutant core protein includes amino acids 1-171 (FIG. 7 (SEQ ID NO: 12)). In another example, the carboxyterminal amino acid is amino acid 178, so that the mutant core protein includes amino acids 1-178 (FIG. 7 (SEQ ID NO: 12)), corresponding to a five amino acid deletion from the carboxyterminus (see, e.g., the analogous duck hepatitis B virus (DHBV) construct pBK, which is described below). The first amino acid sequence is at least 70 amino acids in length, e.g., 72, 74, 76, 78, or 80 amino acids in length. The aminoterminal amino acid of the first amino acid sequence can be the first amino acid of the corresponding wild type hepadnavirus sequence. Alternatively, nonessential aminoterminal amino acids can be eliminated from the mutant polypeptide, provided that the resulting mutant polypeptide does not lose substantial inhibitory activity as a result, when tested according to the methods described below.

By “lacks a second amino acid sequence” is meant that at least three amino acids from the carboxyterminal end of the core protein have been deleted to make the mutant. Preferably, the deleted sequence includes amino acids 171-183 of the HBV core protein; i.e., the second amino acid sequence includes amino acids 171-183 of the sequence shown in FIG. 7 (SEQ ID NO: 12), inclusive.

In another embodiment of the method of inhibiting hepadnavirus replication, the mutant polypeptide further includes a third amino acid sequence. The third amino acid sequence is substantially identical to a portion of a wild type hepadnavirus surface protein. The aminoterminal amino acid of the third amino acid sequence may be joined by a peptide bond to the carboxyterminal amino acid of the first amino acid sequence so as to create a fusion protein. The third amino acid sequence can be the entire surface protein, or can be a portion thereof, e.g., a portion of at least 4, 8, 20, 30, or 43 amino acids in length. For example, the aminoterminal amino acid of the third amino acid sequence can be selected from the group consisting of the amino acids between position 1 and position 112 of the sequence shown in FIG. 8 (SEQ ID NO: 14), inclusive, preferably the amino acids between position 1 and position 8, inclusive. Preferred aminoterminal amino acids of the third amino acid sequence exemplified herein include, but are not limited to, position 5 or position 8 of FIG. 8 (SEQ ID NO: 14).

The carboxyterminal amino acid of the third amino acid sequence can be selected from a group that includes any of the amino acids between position 51 and position 224 of FIG. 8 (SEQ ID NO: 14), inclusive; e.g., any of the amino acids between position 112 and position 224 of FIG. 8 (SEQ ID NO: 14), inclusive; e.g., the carboxyterminal amino acid may be position 51, position 112, or position 224 of FIG. 8 (SEQ ID NO: 14). Thus, the portion of the surface protein included on the mutant polypeptide preferably includes surface protein residues 1-112, 8-112, or 8-51, all inclusive (FIG. 8; SEQ ID NO: 14).

The use of a core protein for inhibiting viral replication is a species-specific event, so that mutant core proteins inhibit nucleocapsid assembly in the same type of hepadnavirus from which they were derived. Thus, the first amino acid sequence is substantially identical to a region of a wild type hepadnavirus core protein that is derived from the same type of hepadnavirus (e.g., HBV versus DHBV) as the naturally-occurring hepadnavirus targeted for inhibition. In contrast, the third amino acid sequence may be substantially identical to a portion of a wild type hepadnavirus surface protein of any hepadnavirus species, since the surface proteins do not demonstrate species specificity. Thus, when the method of the invention is used to treat an HBV infection, the mutant polypeptide should include sequences specifically derived from the HBV core protein FIG. 7 (SEQ ID NO: 12), but can include sequences derived from any species of surface protein (e.g., the sequence of FIG. 8 (SEQ ID NO: 14)).

In another embodiment, the invention features a nucleic acid that encodes a mutant hepatitis B virus (HBV) polypeptide, the polypeptide including a first amino acid sequence that is substantially identical to a region of a wild type HBV core protein, and lacking a second amino acid sequence of the wild type HBV core protein. The second sequence includes the carboxyterminal three amino acids of the wild type HBV core protein and does not exceed nine amino acids in length. Thus, the carboxyterminal amino acid of the first amino acid sequence can be at position 174, position 175, position 176, position 177, position 178, position 179, or position 180, all of FIG. 7 (SEQ ID NO: 12).

In another embodiment, the invention features a nucleic acid that encodes a mutant hepadnavirus polypeptide. The polypeptide includes a first amino acid sequence that is substantially identical to a region of a wild type hepadnavirus core protein; lacks a second amino acid sequence of the wild type hepadnavirus core protein which includes at least the carboxyterminal three amino acids of the wild type hepadnavirus core protein; and includes a third amino acid sequence that is substantially identical to a portion, or all, of a wild type hepadnavirus surface protein. The aminoterminal amino acid of the third amino acid sequence may be joined by a peptide bond to the carboxyterminal amino acid of the first amino acid sequence so as to create a fusion protein. The carboxyterminal amino acid of the first amino acid sequence can be any of the amino acids between position 71 and position 180 of FIG. 7 (SEQ ID NO: 12), inclusive. Preferably, the second amino acid sequence does not exceed 100 amino acids in length.

The invention also features polypeptides encoded by any of the various nucleic acids of the invention. A polypeptide of the invention can be included in a therapeutic composition as an active ingredient, along with a pharmaceutically acceptable carrier, or it can be expressed from the nucleic acid within the infected cell.

The invention also features vectors into which are inserted any of the various nucleic acids of the invention. The vector can include any sequence known to those of skill in the art necessary or desirable for replicating the vector in a eukaryotic cell or for expressing a polypeptide of the invention from the coding sequences thereon. For example, the nucleic acid sequence can be operatively linked to appropriate transcription and/or translation control sequences that function in a eukaryotic cell. The vector can be any vector suitable for maintaining or making multiple copies of a nucleic acid of the invention, or can be one that is suitable for administering a nucleic acid of the invention to a cell or to a mammal infected with a hepadnavirus, e.g., to a human patient infected with HBV or to cells removed from the patient for ex vivo gene therapy. Examples of vectors useful in the method of inhibiting a hepadnavirus include, but are not limited to, adenovirus vectors, adeno-associated vectors, and retroviral vectors. Any of the various vectors of the invention can be included in a therapeutic composition along with a pharmaceutically acceptable carrier.

In another aspect the invention includes a method of evaluating a candidate polypeptide for its ability to inhibit the replication of a naturally-occurring hepadnavirus. The method involves introducing a mutant hepadnavirus polypeptide as described above into a medium in the presence of the hepadnavirus and determining whether hepadnavirus replication is inhibited in the presence of the polypeptide, compared to in its absence, such inhibition being an indication that the polypeptide is an inhibitor of hepadnavirus replication. By “medium” is meant an environment that is capable of supporting viral replication by virtue of its chemical composition. The medium can be within an organism, e.g., an animal model, or can be within an organ removed from an animal. The medium can also be an intracellular medium, e.g., in a cell culture assay, or a cell-free extract, e.g., a cell free replication system. Examples of cells suitable for a cell culture assay include, but are not limited to, Huh-6, Huh-7, HepG2, HepG2 2215, LMH, DC, and HCC cells. The polypeptide can be introduced to the medium by introducing into the medium a nucleic acid encoding the polypeptide, with subsequent expression of the polypeptide therein.

Another method of inhibiting the replication of a naturally-occurring hepadnavirus involves introducing into the proximity of the hepadnavirus a hepadnavirus mutant polypeptide, or a nucleic acid that encodes a hepadnavirus mutant polypeptide. The polypeptide includes a first amino acid sequence that is substantially identical to a region of, or all of, a wild type hepadnavirus core protein, and a second amino acid sequence which is substantially identical to a portion of, or all of, a wild type hepadnavirus surface protein. The aminoterminal amino acid of the second amino acid sequence may be joined by a peptide bond to the carboxyterminal amino acid of the first amino acid sequence so as to create a fusion protein. The second amino acid sequence can be the entire surface protein, or can be a portion thereof. The mutant polypeptide is expressed from the nucleic acid in the proximity of the naturally-occurring hepadnavirus, so as to be available to inhibit replication of the hepadnavirus.

In a final aspect, the invention includes a hepadnavirus mutant polypeptide, or a nucleic acid that encodes a hepadnavirus mutant polypeptide. The polypeptide includes a first amino acid sequence that is substantially identical to a region, or all, of a wild type hepadnavirus core protein, and a second amino acid sequence which is substantially identical to a portion, or all, of a wild type hepadnavirus surface protein. The aminoterminal amino acid of the second amino acid sequence may be joined by a peptide bond to the carboxyterminal amino acid of the first amino acid sequence so as to create a fusion protein. The second amino acid sequence can be the entire surface protein, or can be a portion thereof.

As used herein, a “hepadnavirus” refers to a member of the hepadnavirus family of viruses, including, but not limited to, hepatitis B virus and hepatitis delta virus (Wang et al., Nature, 323:508-13, 1986). Although treatment of HBV is an important feature of the method of invention due to the incidence of HBV-related human disease, the methods described herein also apply to other species of hepadnaviruses. Examples of hepadnaviruses within the scope of the invention include, but are not limited to, hepadnaviruses infecting various human organs, including liver cells, exocrine and endocrine cells, tubular epithelium of the kidney, spleen cells, leukocytes, lymphocytes, e.g., splenic, peripheral blood, B or T lymphocytes, and cells of the lymph nodes and pancreas (see, e.g., Mason et al., Hepatology, 9:635-645, 1989). The invention also applies to hepadnaviruses infecting non-human mammalian species, such as domesticated livestock or household pets. In addition, the invention includes a method of evaluating a candidate mutant polypeptide for its ability to inhibit hepadnavirus replication. For the purposes of conducting a laboratory screening assay, a variety of hepadnavirus species are useful models. Examples include, but are not limited to, woodchuck hepatitis virus (WHV; Summers et al. Proc. Natl. Acad Sci. USA, 75:4533-37, 1978), duck hepatitis B virus (DHBV; Mason et al. J. Virol. 36:829-36, 1978), and squirrel hepatitis virus (e.g., Marion et al. Proc. Natl. Acad Sci. USA, 77:2941-45, 1980).

Although particular amino acids are referred to below with reference to the sequence of HBV (FIGS. 7 and 8; SEQ ID NOs: 11-14), it is understood that the invention encompasses mutant polypeptides comprising corresponding amino acid segments derived from other hepadnavirus species. One of ordinary skill in the art can easily compare closely-related sequences to locate the analogous amino acid positions in related hepadnaviruses; the descriptions provided in Examples 2 and 3 illustrate examples of such comparisons.

Where the method of inhibiting hepadnavirus replication is used to treat a hepadnaviral infection in an animal, a “naturally-occurring” hepadnavirus refers to a form or sequence of the virus as it exists in an animal, e.g., a natural isolate derived from an infected animal. In all other contexts, a “naturally-occurring” hepadnavirus is intended to be synonymous with the sequence known to those skilled in the art as the “wild type” sequence, e.g., the wild type HBV core and surface protein sequences shown in FIGS. 7 and 8 (SEQ ID NOs: 11-14). If an amino acid sequence of a core or surface protein of a hepadnavirus that is derived from a natural isolate differs from the conventionally accepted “wild type” sequence, it is understood that the sequence of the natural isolate may be the proper comparison sequence for designing mutant polypeptides of the invention. “Sequence identity”, as used herein, refers to the subunit sequence similarity between two nucleic acid or polypeptide molecules. When a given position in both of the two molecules is occupied by the same nucleotide or amino acid residue, e.g., if a given position in each of two polypeptides is occupied by serine, then they are identical at that position. The identity between two sequences is a direct function of the number of matching or identical positions, e.g., if half (e.g., 5 positions in a polymer 10 subunits in length) of the positions in two polypeptide sequences are identical, then the two sequences are 50% identical; if 90% of the positions, e.g., 9 of 10, are matched, the two sequences share 90% sequence identity. Methods of sequence analysis and alignment for the purpose of comparing the sequence identity of two comparison sequences are well known by those skilled in the art. By “substantially identical” is meant sequences that differ by no more than 10% of the residues, and only by conservative amino acid substitutions, such as shown in Table 1 of U.S. Ser. No. 09/372,548 (from which the present application is a continuation), or non-conservative amino acid substitutions, deletions, or insertions that do not appreciatively diminish the polypeptide's biological activity, e.g., an insertion of amino acids at the junction of the core protein and surface protein sequences that has no appreciative effect on biological activity. “Biological activity”, as used herein, refers to the ability of a mutant polypeptide to inhibit hepadnavirus replication, and can be measured by the assays described below.

Other terms and definitions used herein will be understood by those of routine skill in the art. For example, by “inhibiting the replication of” is meant lowering the rate or extent of replication relative to replication in the absence of a mutant polypeptide of the invention. By “into proximity with the hepadnavirus” is meant introducing into a cell, organ, or organism which is infected with a naturally-occurring hepadnavirus, or, in the case of laboratory application, cotransfection or co-inoculation with a wild type hepadnavirus. By “nucleic acid” is meant deoxyribonucleic acid (DNA) or ribonucleic acid (RNA).

The methods, nucleic acids, and polypeptides of the invention can be used to inhibit hepadnaviral replication in a mammal, e.g., as an effective therapy for treating individuals with a persistent HBV infection, or as a means of reducing the risk of hepatocellular carcinoma in an infected animal. Polypeptides of the invention can be administered to an infected animal either directly or by gene therapy techniques. The screening methods of the invention are simple, rapid, and efficient assays designed to identify polypeptides with anti-hepadnaviral activity.

Other features and advantages of the invention will be apparent from the following detailed description and from the claims.

### BRIEF DESCRIPTION OF THE DRAWINGS

FIG. 1 is a schematic illustration of the structural organization of “wild type” and mutant hepadnavirus constructs.

FIG. 2 is an illustration of the nucleic acid sequence of the pCN4 plasmid insert (SEQ ID NO: 1) and the corresponding translated amino acid sequence (SEQ ID NO: 2).

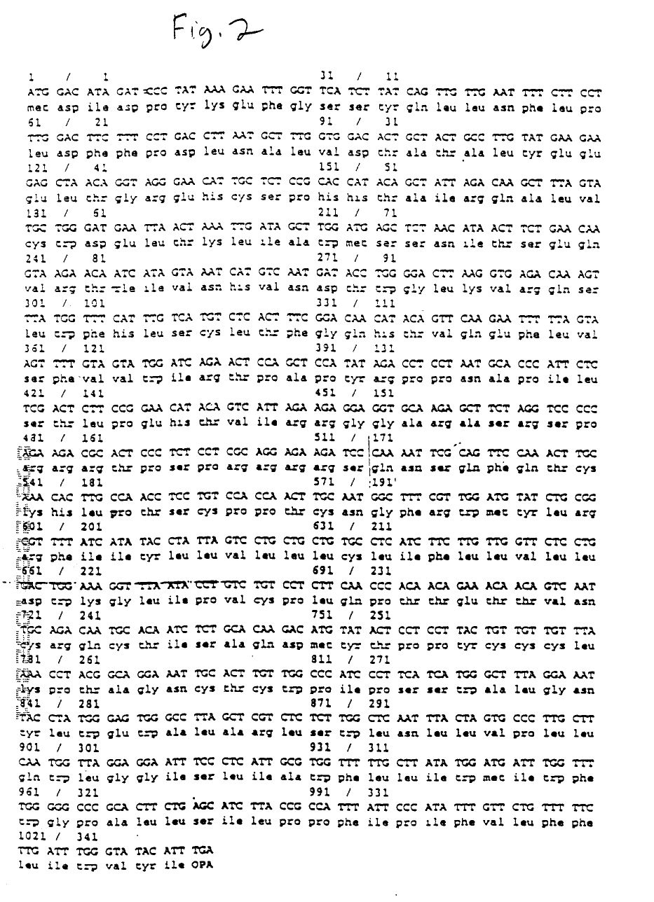

FIG. 3 is an illustration of the nucleic acid sequence of the pHBV DN plasmid insert (SEQ ID NO: 3) and the corresponding translated amino acid sequence (SEQ ID NO: 4).

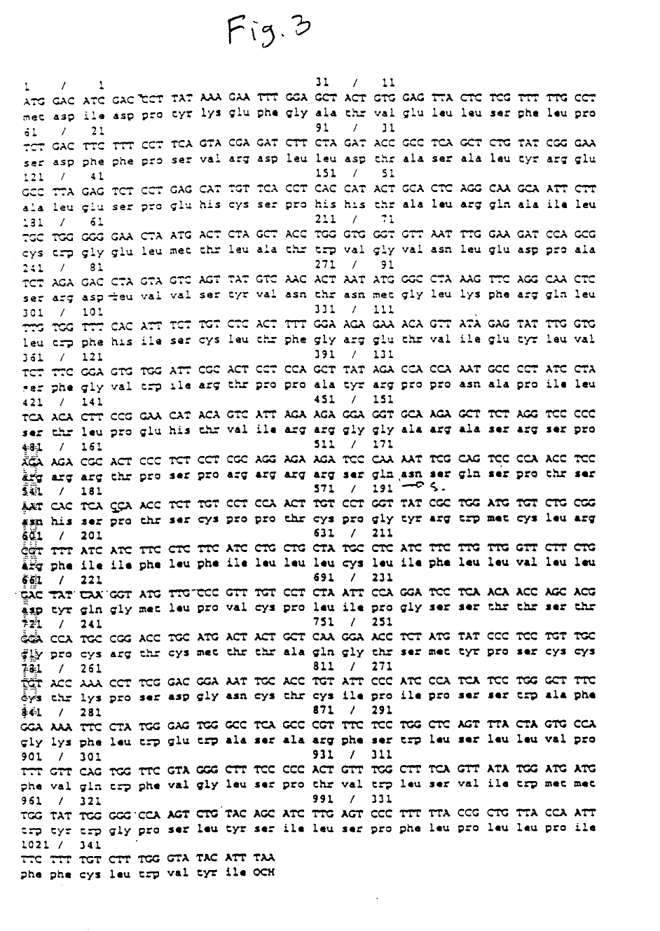

FIG. 4 is an illustration of the nucleic acid sequence of the pHBV DN AA plasmid insert (SEQ ID NO: 5) and the corresponding translated amino acid sequence (SEQ ID NO: 6).

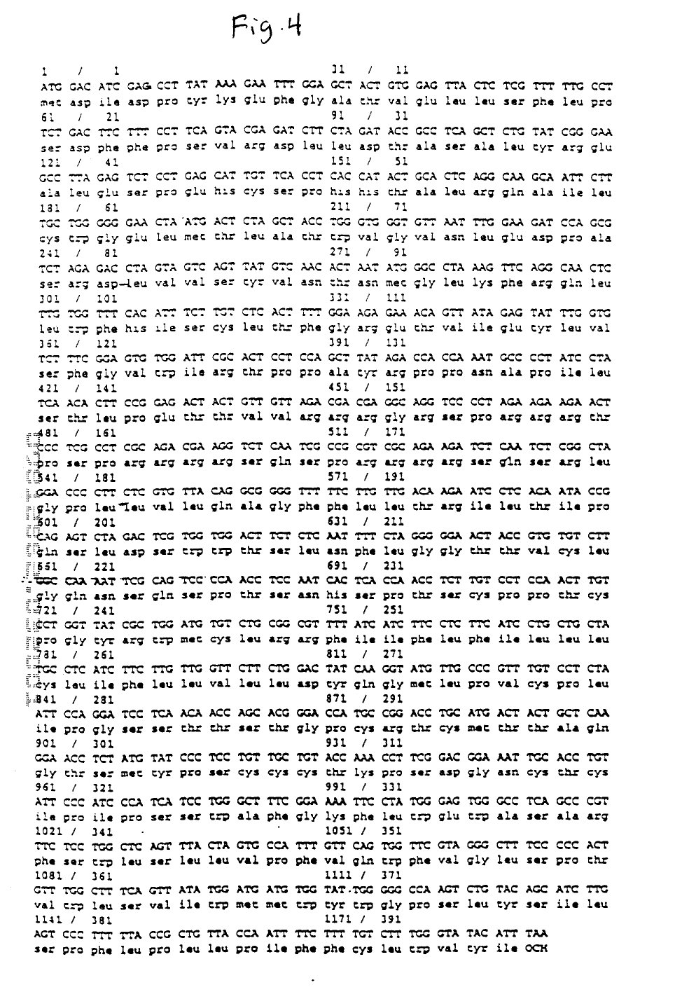

FIG. 5 is an illustration of the nucleic acid sequence of the pHBV DN BB plasmid insert (SEQ ID NO: 7) and the corresponding translated amino acid sequence (SEQ ID NO: 8).

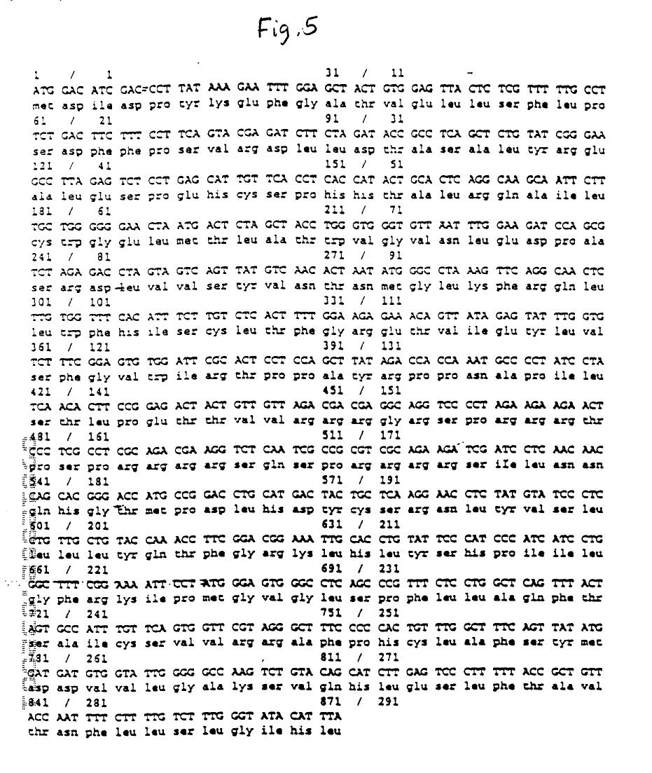

FIG. 6 is an illustration of the nucleic acid sequence of the pDHBV BK plasmid insert (SEQ ID NO: 9) and the corresponding translated amino acid sequence (SEQ ID NO: 10).

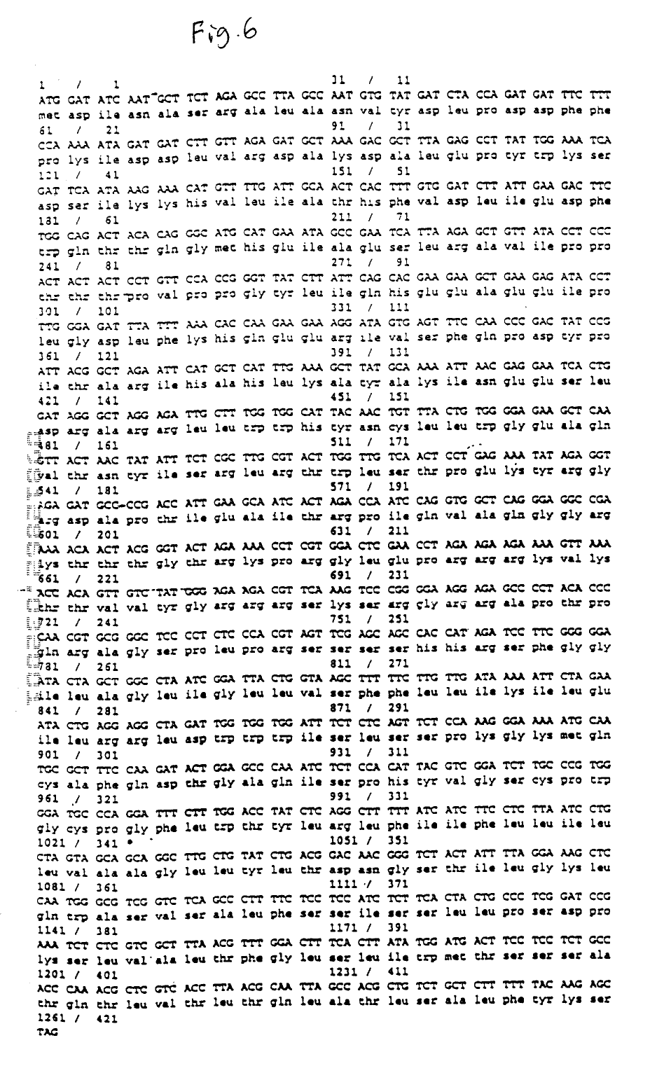

FIG. 7 is an illustration of the nucleic acid sequence of the HBV core protein (SEQ ID NO: 11) and the corresponding translated amino acid sequence (SEQ ID NO: 12).

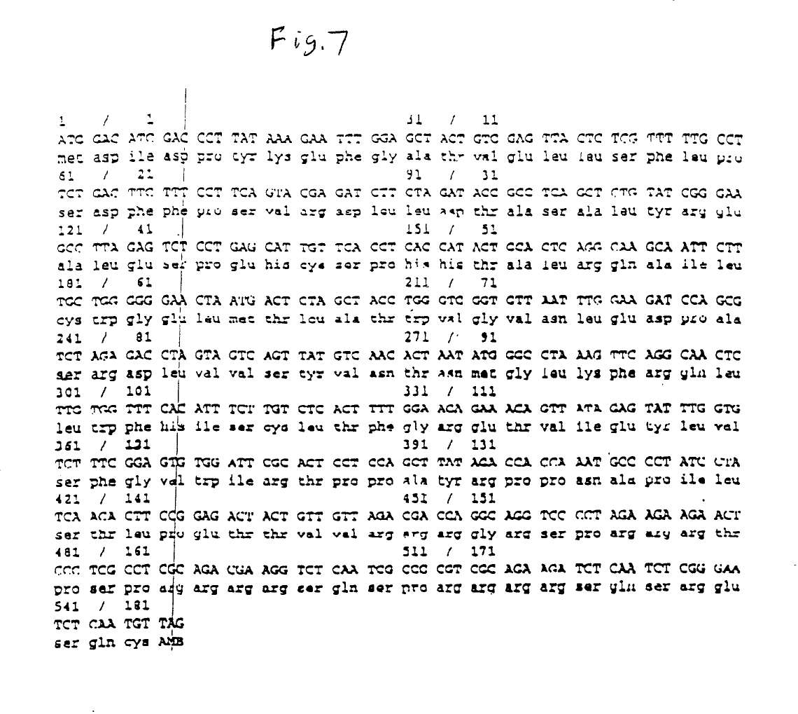

FIG. 8 is an illustration of the nucleic acid sequence of the HBV core protein (SEQ ID NO: 13) and the corresponding translated amino acid sequence (SEQ ID NO: 14).

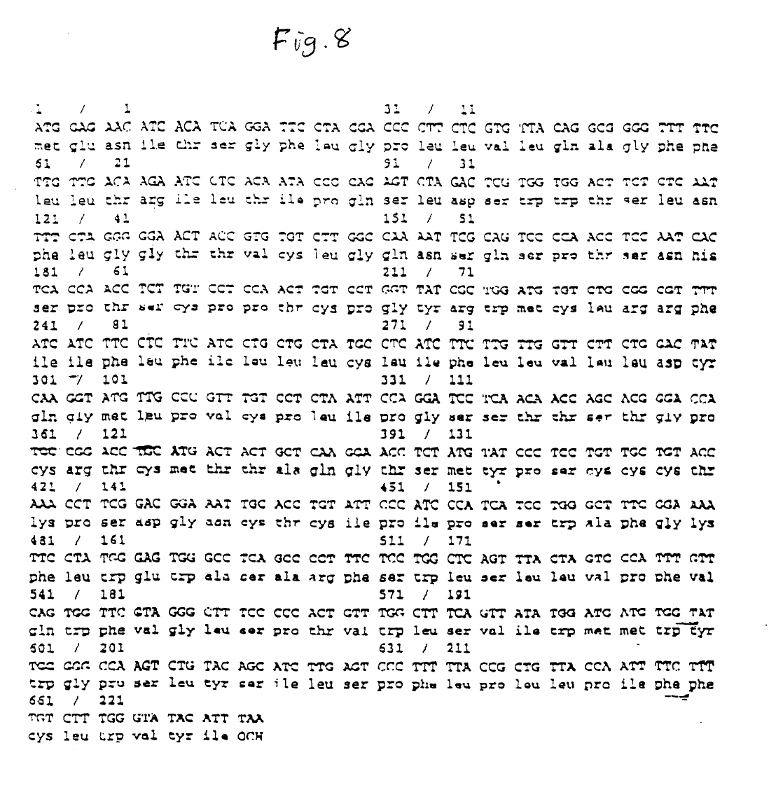

### DETAILED DESCRIPTION OF THE INVENTION

Applicants have observed that replication of a wild type hepadnavirus is reduced when it is co-transfected with a nucleic acid construct encoding a truncated core protein, or a core-surface fusion protein. The truncated core protein, alone or in combination with the surface protein component, has a deletion of at least three amino acids from the carboxyterminal end. Viral replication is reduced by as much as 90-95% without detectable toxic effects on the host cell. Constitutively expressing a HBV mutant core-surface fusion protein as a retroviral insert substantially inhibits HBV viral DNA production in cells that previously had continuously produced all viral replicative intermediates and infectious virions. An adenoviral-based plasmid that encodes the same mutant core-surface fusion protein also inhibits HBV replication following transient cotransfection in HCC cells. These dominant negative effects on viral replication are consistent over a range of hepadnavirus species.

Materials and Methods

Materials and methods useful for practicing the invention are described as follows:

Plasmid Constructs.

The parental plasmid pCMW82 was used to generate a series of constructs expressing WHV core proteins with an altered carboxyterminal region. Plasmid pCMW82 expresses the “wild type” WHV pregenome under the control of the cytomegalovirus immediate-early (CMV IE) promoter (Seeger et al., J. Virol., 63, 4665-4669, 1989). The pHBV plasmid carries the HBV pregenome under the control of the CMW IE promoter. These plasmids direct the synthesis of complete virions in tissue culture cells. The first nucleotide of the precore open reading frame was designated as nucleotide number 1 in the WHV genome.

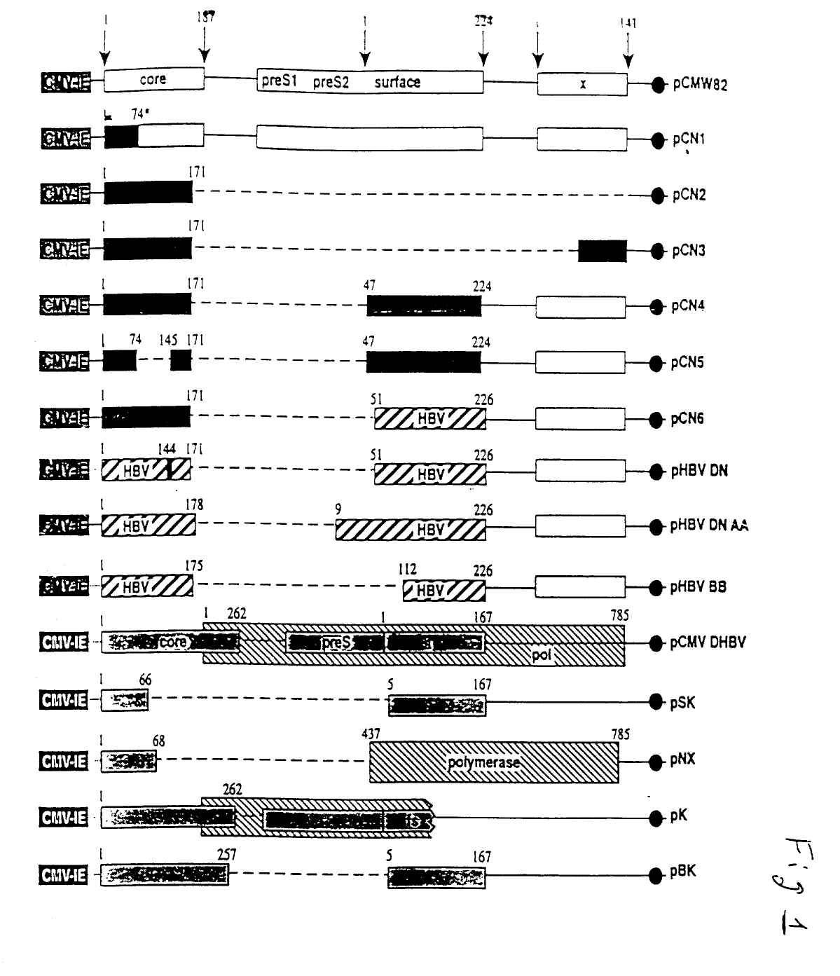

The structural organization of “wild type” and mutant WHV, HBV, and DHBV core plasmid constructs are depicted in FIG. 1. The white boxes represent the open reading frame (ORF) used for constructing core mutants. Numbers at the boundaries of each ORF refer to the amino acids in the “wild type” or mutant proteins. Dotted lines represent deleted sequences. Solid and hatched boxes correspond to mutant core proteins expressed from WHV and HBV, respectively. Shaded bars refer to DHBV. The shaded hatched bars refer to the polymerase gene. Except for the “wild type” constructs pCMW82 and pCMW-DHBV, all other constructs are incapable of replication because of deletions in genes that overlap the truncated portions of the core protein. The * refers to a stop codon introduced by a frame shift mutation.

The constructs shown in FIG. 1 were produced by complete digestion with the appropriate restriction enzyme. This was followed by subsequent incubation at 30° C. for 20 min. in the presence of the Klenow fragment of DNA polymerase I and deoxyribonucleoside triphosphates, which filled in the 3′ recessed DNA ends. Plasmids were then ligated with T4 DNA ligase. The 3′ protruding ends were filled in by incubation with the Klenow fragment of DNA polymerase I in the absence of deoxyribonucleoside triphosphates at 37° C. for 15 min. This eliminated protruding ends. Deoxyribonucleoside triphosphates were then added and incubation was carried out at 30° C. for 20 min. (Sambrook et al., Molecular Cloning: A Laboratory Manual, Cold Spring Harbor Laboratory, Cold Spring Harbor, N.Y., 1989).

Constructs containing mutations in sequences encoding the core protein were obtained as follows: 1) pCN1: To make the plasmid pCN1 the WHV core gene was digested with the restriction enzyme SstI at nucleotide (nt) 310, incubated with Klenow DNA polymerase, and reclosed with T4 DNA ligase. This introduced a frame shift mutation at nt. 306 in the WHV core gene, thereby creating a stop codon at nt. 317. This mutation produces a 74 amino acid carboxyterminal truncated core protein, leaving intact the rest of the viral coding regions. 2) pCN2: To make pCN2 the WHV parental plasmid was digested with the restriction enzymes BglII (nt. 601 in the core gene) and SmaI, the latter being located in the downstream multiple cloning site of the vector. The intervening viral genes were separated by gel electrophoresis, and the DNA ends were filled in with Klenow DNA polymerase and ligated with T4 DNA ligase. This WHV core gene has 12 amino acids deleted at the carboxyterminus and is fused to a three amino acid heterologous extension from the plasmid vector. 3) pCN3: To make the plasmid pCN3, the wild type plasmid pCMW82 was digested with the restriction enzymes BglII and SacII (position 2983 in the WHV X gene), the intervening viral DNA fragment was removed, and ends were filled in and ligated. The resulting plasmid construct encodes a 171 amino acid core protein fragment fused in-frame with the X protein at amino acid 31. 4) pCN4: The plasmid pCN4 was produced by a BglII-MscI (position 1826) fragment excised from pCMW82 and blunted by Klenow DNA polymerase. The plasmid was ligated to join the WHV 171 amino acid core protein as an in-frame fusion protein with amino acid 47 of the WHV small surface protein. 5) pCN5: The plasmid pCN5 was produced by removing the DNA fragment SstI (pos. 306)-BspEI (pos. 519) from pCN4, and blunting the ends with Klenow DNA polymerase and T4DNA ligase. This introduced a WHV core in-frame deletion between amino acids 74 and 145. 6) Plasmid pCN6 expresses the first 171 amino acids of the WHV core protein fused in-frame with the HBV small surface protein at amino acid 51.

The HBV numbering system designates the unique EcoRI site as nucleotide 1. Construct pHBV DN was generated by digesting pCMW82 at nt. 601 of the core gene with BglII, and blunting the DNA end with Klenow DNA polymerase. A second cut was performed with PvuI in the ampicillin resistance gene of the carrier plasmid, and the BglII-Pvul DNA fragment was removed by fractionation on an agarose gel. The HBV MscI (pos. 299)-PvuI (in the ampicillin resistance gene of pHBV) fragment was ligated to the blunted BglII-PvuI fragment.

In order to produce an in-frame dominant negative construct of HBV that was similar to the pCN4 WHV construct, the pCN6 fragment from the SnaBI site (which cuts in the CMV promoter of the carrier plasmid) to the BspEI site (pos. 519 in the WHV) was removed and substituted with the same SnaBI-BspEI (pos. 2327) fragment from pHBV. In this way, the HBV core protein was fused in-frame to amino acid 144 of the WHV core protein. This fragment, derived from plasmid pCN6, was already fused at amino acid 171 to the HBV small surface protein at amino acid 51. The resulting pHBV DN therefore encodes, in the hinge between the deleted core and surface proteins, five amino acids derived from the WHV core protein (GGARA). These five amino acids were not present in the subtype HBV core protein. The carboxyterminal 20 amino acid of the WHV core protein are conserved in HBV.

Two additional plasmids were derived from pHBV and called pHBV AA and pHBV BB. To make pHBV DN AA, pHBV was partially digested with the restriction enzyme AvaI (nt. 2431), and then partially digested with AvrII (nt. 176). The resulting DNA ends were blunted by adding Klenow DNA polymerase and nucleotide triphosphates. The DNA ends were ligated with T4 ligase. The resulting plasmid pHBV DN AA encodes the HBV core protein fused in frame at amino acid 179 with the surface protein (encoded by the “S gene”) at amino acid 8. The plasmid pHBV BB was made by performing two sequential partial digestions with the enzymes BglII and BamHI. The DNA ends were ligated with T4 ligase. The pHBV BB plasmid expresses the HBV core protein fused in frame at amino acid 175 with the surface protein at amino acid 112. The correct design of the constructs was confirmed by restriction digest mapping and DNA sequence analysis. Plasmid DNAs were purified by the alkali lysis procedure followed by sedimentation through a cesium chloride-ethidium bromide density gradient. As a result of these viral gene manipulations the above plasmid constructs produce replication defective WHV genomes. Plasmid pCN1 expresses a truncated core protein that is unable to assemble into functional nucleocapsids. All other constructs contain inactivating deletions in the polymerase gene.

Another plasmid, designated pRHBBE, was constructed using the polylinker of the plasmid pBS SK(+)(Stratagene), which allows for viral gene transcription from the T7 promoter to make a HBV-specific 276 nt antisense RNA. This species, encoded by a BamHI (pos. 2906) to EcoRI (pos. 1) fragment, was used in RNase protection experiments. The 32P labeled riboprobe annealed specifically to the “wild type” pregenomic HBV DNA without recognizing the pHBV DN mRNA.

Constructs expressing DHBV dominant negative proteins were derived from the plasmid pCMV DHBV (Wu et al., J. Virol., 65, 2155-2163, 1991), which expresses the DHBV pregenome under the control of the CMV promoter. Construct pSK contains a deletion between the SphI site (position 2843 in the core gene; this numbering system is arbitrarily initiated with the nucleotide GAATTC of the unique EcoRI site) and the KpnI site (position 1290, in the S gene). The intervening fragment was separated by agarose gel electrophoresis. The ends of the larger DNA fragment were blunted by Klenow DNA polymerase and religated. This construct expresses, under the control of the CMV promoter, a protein composed of the first 66 amino acids of the DHBV core protein fused in frame to amino acid five of the DHBV surface protein. Construct pBK contains a deletion between the BglII site (position 391 in the core gene) and the KpnI site (position 1290 in the S gene). The intervening fragment was separated by agarose gel electrophoresis and the ends of the larger DNA fragment were filled in and blunted by the Klenow DNA polymerase. The ends were then religated. The resulting construct expresses, under the control of the CMV promoter, a protein composed of the first 257 amino acids of the DHBV core protein (five amino acids are missing from the carboxyterminus), fused in frame to the fifth amino acid of the DHBV surface protein.

To make the construct pK, the pCMV DHBV was linearized by cutting at the KpnI site (position 1290 of the S gene). The DNA ends were blunted with the Klenow DNA polymerase reaction and the fragment was religated. The resulting construct has a frame-shift mutation so that the DHBV polymerase pK gene and the pre-S and S genes have a termination site a few nucleotides downstream from the KpnI site. The construct pK thus expresses, under the control of the CMV promoter, the full length core protein, but none of the envelope proteins apart from a truncated pre-S protein. A frameshift mutation that occurs in the polymerase gene renders the other constructs carrying the deletions described above replication defective. Construct pNX contains a deletion between the NsiI site (position 2845 in the core gene) and the XhoI site (position 1212 in the pre-S gene). The intervening fragment was separated by agarose gel electrophoresis. The ends of the larger DNA fragment were blunted and filled in with Klenow DNA polymerase, followed by religation of the fragment to itself. This construct expresses, under the control of the CMV promoter, the first 68 amino acids of the DHBV core protein fused in frame to amino acid 437 of the carboxyterminus of the polymerase gene.

Retroviral Constructs.

The HBV core-surface fusion gene encoded by pHBV DN was PCR amplified with oligonucleotides containing at their 5′ ends a SalI restriction enzyme recognition site. The antisense primer contained a recognizable Flag epitope (Kodak). The PCR product was gel purified, digested with SalI, and cloned in the retroviral pBabe Puro vector (Morgenstern et al., Nucl. Acids Res., 18:3587-96, 1990) at its SalI site. The design of the resulting pBP HBV DN vector was confirmed by sequence analysis.

Transfections into Hepatoma Cell Lines.

Human hepatoma cells HuH-7 and HepG2 support a complete viral replicative cycle following transfection with a plasmid construct expressing the pregenomic viral RNA (Mason et al., Hepatology, 9, 635-45, 1989). Cells were maintained and passaged as previously described (Wu et al., J. Virol., 65, 2155-2163, 1991). Cells were transiently co-transfected with plasmids expressing the mutated WHV or HBV core genes (described above), together with an equal amount of a “wild type” WHV or HBV producing plasmid. Co-transfections were performed by the calcium phosphate technique (CaPO₄ transfection Kit, 5′-3′, Boulder, Colo.). Briefly, 1.2×10⁷ cells in 100 mm plates were grown for 24 hours. The medium was changed 2-4 hours before transfecting with 10 μg of “wild type” virus. This produces the plasmid along with 10 μg of each mutant construct. The precipitate was left on the cells for 6-8 hours, and then the medium was replaced. The cells were harvested two days after transfection when performing RNA experiments, and five days after transfection when performing DNA experiments.

The cell line LMH, derived from a chicken hepatocellular carcinoma, was used for transfection of the DHBV derived plasmids. This cell line supports higher levels of DHBV replication than do cell lines of human origin. Another cell line, derived from LMH and named D2, was created by stably transfecting a head-to-tail DHBV dimer that produces infectious DHBV particles. These cells were grown in DMEM and 10% FCS and transfected with the various dominant negative core mutant constructs as described above.

Infection of the HepG2 2215 Cell Line.

Infection of the HepG2 2215 cell line by recombinant retroviruses was accomplished following a standard protocol for producing retroviral stocks and for infecting tissue culture cells (Miller et al., Biotechniques, 7, 980-990, 1989; Miller, et al., Methods in Enzymology, 217, 581, 1993). After infection, the cells were selected with 2 μg/ml puromycin (Sigma). Resistant clones were pooled and further expanded.

Analysis of Viral DNA Replication.

WHV and HBV DNA replication were assayed by Southern blot analysis of DNA that had been extracted from intracellular core particles. The procedure for isolation of core particles was previously described in detail (Pugh et al., J. Virol., 62, 3513-3516, 1988). DNA fractionation on agarose gels was performed under alkali conditions and the DNA was transferred onto a nylon membrane for Southern blot analysis (Hybond N, Amersham International, Little Chalfont, UK). Prehybridization and hybridization reactions were carried out at 65° C. in 6X SSC solution (1X SSC is 150 mM NaCl, 15 mM Na₃Citrate), 5X Denhardt's solution (100X is 2% w/v BSA, 2% w/v Ficoll, 2% w/v polyvinyl pyrrolidone), and 0.5% SDS. WHV and HBV DNAs were detected by hybridization with randomly primed ³²P-labeled full length WHV or HBV DNA (Multiprime DNA Labeling System, Amersham). The membranes were washed twice for 15 min. each at 65° C. in 1X SSC, 0.1% SDS, and were then washed once more at 65° C. in 0.1X SSC, 0.1% SDS. The nylon membranes were then autoradiographed at −70° C., using intensifying screens and Kodak films. Signal intensities on the nylon sheets were quantitated by a computer assisted scanning system (Ambis Quantprobe System version 3.0).

Extraction and Analysis of Viral RNA.

Total RNA was extracted from a 100 mm dish by lysis of cells in 1 ml of solution D (4 M guanidinium thiocyanate, 25 mM NaCitrate, pH 7.0, 0.5% sarcosyl, 0.1 M 2-mercaptoethanol) as described (Chomczynski et al., Anal. Biochem., 168, 156-159, 1987). Encapsulated viral RNA was extracted from core particles by lysis in 200 ml of solution D and the volumes were adjusted accordingly as described (Roychoury et al., supra). Finally, to exclude contamination by plasmid DNA or reversed transcribed HBV DNA, the encapsulated viral RNA was digested with 16 U RNase-free DNase RQ1 DNase (Promega, Madison, Wis.) at 37° C. for 15 min., followed by phenol-chloroform extraction and ethanol precipitation, before undergoing the RNase protection assay.

RNase protection analysis of total and encapsulated viral RNA was performed with a commercially available kit according to the manufacturer's instructions (RPA II-Ribonuclease protection kit, Ambion Inc. Austin, Tex.). The RNA probe was derived from the plasmid pRHBBE, a derivative of the pBluescript SK(+), which includes the 280 bp HBV fragment BamHI (pos. nt 2901)-EcoRI (pos. nt 1), oriented to produce an antisense RNA molecule when transcription was initiated with the bacteriophage T7 RNA polymerase. The RNA probe contained approximately 50 nt of plasmid sequences that were not protected by the HBV specific RNA. Labeled RNA was synthesized as follows: 0.5 μg of pRHBBE was cut by BamHI and then transcribed by T7 RNA polymerase (Promega, Madison, Wis.) in the presence of α-³²P UTP (100 μCi at 400 Ci/mM, New England Nuclear, Boston, Mass.). The antisense RNA probe recognized pregenomic RNA and the 2.4 pre-S1 mRNA derived from “wild type” HBV, but did not recognize transcripts derived from pHBV DN. Hybridization, after denaturation at 95° C. for 3 min., was performed in 20 μl on 2 μg of total RNA or encapsulated pregenomic RNA derived from half of a 100 mm plate at 42° C. overnight in a solution of 80% formamide, 100 mM NaCitrate pH 6.4, 300 mM NaAcetate pH 6.4, and 1 mM EDTA. RNase digestion was carried out with RNase A (0.5U) and RNase Ti (20 U) at 37° C. for 30 min. Fragments protected by RNase digestion were separated on a denaturing 6% polyacrylamide gel (Sequagel 6%, National Diagnostics, Atlanta, Ga.).

Viral nucleocapsid isolation and Western blots.

HepG2 cells that had been transfected with pHBV alone, pHBV DN together, or pHBV DN alone were lysed in 500 ml TNE, 1% NP 40. The debris was pelleted by centrifugation at 10,000 rpm in an Eppendorf bench top centrifuge. A 200 μl aliquot of the clarified cell lysate was ultracentrifuged at 500,000× g for 1 hour at 4° C. through 2 ml of a 20% w/v sucrose/TNE cushion using a TLA 100 rotor (Beckman Instruments, Palo Alto, Calif.). Under these conditions viral core particles were pelleted, whereas free core protein and soluble hepatitis Be antigen (HBeAg) remained in the supernatant (Zhou et al., supra). The pellet was resuspended in 100 μl of Laemmli sample buffer and boiled for 3 min. One-half of the sample was run over a 12.5% SDS-PAGE gel (Acrygel National Diagnostics, Atlanta, Ga.). Western blotting was performed on an Immobilon-P membrane (Millipore Co., Bedford, Mass.) (Harlow et al., Antibodies: a laboratory manual, Cold Spring Harbor Laboratories, CSH, N.Y. 1988). After transfer the membrane was blocked for one hour in a solution of 5% non-fat dry milk and 0.5% Tween-20 in phosphate buffered saline (PBS). HBcAg antigenicity was detected by incubation of the membrane with polyclonal antibodies prepared in rabbits against recombinant HBcAg (Dake Co., Carpinteria, Calif.) at a 1:250 dilution in the above solution for one hour at 20° C. The filter was washed at 20° C. in PBS, 0.5% Tween-20 with three successive changes of solution. Bound antibody was detected using the chemiluminescence method (ECL, Amersham International, Little Chalfont, UK) using peroxidase labeled goat anti-rabbit antibodies. The filter was exposed to Kodak films for 5-20 seconds.

Experimental Results

Inhibition of WHV DNA Synthesis.

WHV core mutant plasmids were tested for the ability to inhibit wild type WHV DNA replication in HuH-7 cells. Core particle DNA was extracted from HuH-7 cells five days post transfection and probed with full length ³²P labeled WHV DNA in a Southern blot. Lane M contains P 5′ end labeled lambda HindIII molecular weight markers. The HuH-7 cells were transfected with: lane 1, pCMW82; lane 2, pCMW82 and pCN4; lane 3, pCMW82 and pCN1; lane 4, pCMW82 and pCN2; lane 5, pCMW82 and pCN3; and lane 6, pCMW82 and pCN5. Each lane was loaded with one-half of the core associated viral DNA, which had been extracted from a 100 mm tissue culture dish of HuH-7 cells.

All mutant WHV core constructs suppressed “wild type” WHV DNA synthesis, albeit with different efficiencies. The extent of inhibition varied among the different constructs, depending in part on the molecular structure of the mutant core protein. In order to exclude experimental variability, all transfections were repeated several times with comparable results. The data represent an average of at least three independent experiments. Cotransfection of “wild type” pCMW82 with the mutant core constructs pCN1, pCN2, and pCN3 produced a modest inhibition of “wild type” viral DNA replication (36%, 48%, and 12%, respectively). In contrast, pCN4 and pCN5substantially inhibited WHV DNA synthesis in HuH-7 cells by 90% and 85%, respectively.

To test whether the pCN4 construct inhibits HBV replication, cotransfection experiments were performed with “wild type” pHBV. There was no reduction of HBV synthesis by the WHV based construct pCN4. The analysis included a Southern blot of core particle associated viral DNA extracted from HuH-7 cells five days after transfection. The blots were probed simultaneously with full length ³²P labeled WHV and HBV DNA probes. Lane M contains ³²P 5′ end labeled lambda HindIII molecular weight markers. Core particle associated viral DNA was extracted from cells transfected with: lane 1, pCMW82; lane 2, pCMW82 and pCN4; lane 3, pCMW82 and pCN6; lane 4, pHBV; lane 5, pHBV and CN4; and lane 6, pHBV and pCN6. Each lane was loaded with one-half of the core particle associated DNA that had been extracted from a 100 mm tissue culture dish of HuH-7 cells.

In order to determine the general region of the fusion protein that was responsible for inhibiting viral replication, a chimeric construct expressing WHV core-HBV small surface fusion protein was produced. This plasmid, designated pCN6, reduced “wild type” WHV replication by 85%, an inhibitory effect comparable to the original parental construct pCN4. Like pCN4, pCN6 does not inhibit HBV replication. It was concluded that the WHV core-small surface fusion protein encoded by pCN4 exerts a species-specific inhibitory effect.

To determine the amount of pCN4 required to interfere effectively with WHV replication, HuH-7 cells were co-transfected at various ratios of CMW82 to pCN4 using 10 μg of pCMW82. The total amount of transfected DNA was kept constant (20 μg) by adding unrelated sonicated salmon sperm DNA. The results of these experiments indicate that when pCN4 was diluted by 10 and 50 fold, there was still a 66% and 20% inhibition of “wild type” WHV replication, respectively. Interference with viral replication occurs even in the presence of an excess of “wild type” core protein.

Dominant negative core mutant polypeptides are not toxic to HCC cells. To insure that the mutant plasmids were neither affecting the efficiency of DNA uptake by HuH-7 cells during transfection, nor inducing a cytopathic effect, each 100 mm plate had a 10 mm cover slip containing cells grown under the same conditions. The cells were investigated by immunocytochemistry utilizing the protocol of Jilbert et al. (J. Virol., 66, 1377-1378, 1992). Core protein expression was detected with polyclonal antibodies prepared against either WHV or HBV recombinant core proteins. Approximately one percent of the HuH-7 cells were transfected with the “wild type” WHV plasmid, as demonstrated by diffuse cytoplasmic staining for WHcAg in cells harvested five days post transfection. After transfection of cells with pCN4 alone, a punctate distribution of WHcAg in the perinuclear region was observed. The same staining pattern was obtained when the dominant negative core mutant constructs were co-transfected with “wild type” pCMW82. The total number of HBcAg positive cells did not vary under these conditions. The mutant core expressing plasmids did not inhibit “wild type” viral DNA uptake during the transfection process and were not toxic to HuH-7 cells.

It was also necessary to exclude the possibility that the inhibitory effect exerted by pCN4 on WHV replication was the result of decreased transcription of “wild type” WHV pregenomic RNA. For these studies, Poly(A)+RNA was extracted from HuH-7 cells that had been transfected with the plasmids pCMW82 alone, pCMW82 and pCN4 together, or pCN4 alone. The RNA was probed with a BglII-BstXI WHV DNA fragment that specifically recognized the pregenomic RNA but not the pCN4 transcripts. The results demonstrated no change in the level of “wild type” WHV pregenomic RNA transcription from pCMW82 in the presence of pCN4.

Inhibition of HBV Replication.

Based on the previous results, it was of interest to determine whether a similar mutant core polypeptide would inhibit HBV replication in HCC cells. The construct pHBV DN was designed to be the molecular HBV-derived equivalent of pCN4. Plasmid pHBV DN was co-transfected with “wild type” pHBV into HuH-7 and HepG2 cells. It inhibited “wild type” HBV DNA replication by 90%.

Included in the analysis immediately above was a Southern blot of core particle associated viral DNA extracted from HepG2 cells five days after transfection. The blot was probed with full length ³²P labeled HBV DNA. Lane M contains ³²P 5′ end labeled lambda HindIII molecular weight markers. Lane 1 contains 3.2 kb linear HBV DNA (10 pg). The remaining lanes show core particle associated viral DNA extracted from cells transfected with pHBV (lane 2); or PHBV and pHBV DN (lane 3).

The constructs pHBV DN AA and pHBV DN BB were assayed in the same manner, for the purpose of mapping which regions of the core protein and of the surface protein were necessary for inhibition. The construct pHBV DN AA was at least as potent an inhibitor as pHBV DN, whereas pHBV DN BB was less inhibitory than pHBV DN. Included in this analysis was a Southern blot analysis illustrating the antiviral effects of the pHBV DN AA and pHBV DN BB dominant negative core mutants on “wild type” HBV replication during transient transfection experiments in HuH-7 cells. The pCMV-HBV lane shows the level of “wild type” HBV replication in HUH-7 cells. The dominant negative mutant pHBV-DN reduced wild type replication by 95%. When this construct was placed in a vector containing the adenovirus sequences necessary for producing a recombinant adenovirus vector (Ad HBV DN), there was an 80% decrease in HBV replication. When the HBV DN construct was placed in a retroviral vector (e.g., pBP HBV DN), there was a 90-95% reduction in HBV replication.

Experiments were then performed to assess the presence and amount of pregenomic RNA within nucleocapsids, and to compare these results to the level of viral RNA present in the cytosolic fraction by means of a sensitive RNase protection assay. RNA was extracted from HepG2 transfected cells and probed with a ³²P labeled 322 nt RNA probe containing the BamHI (pos. 2906)-EcoRI (pos. 1) fragment (lane P), or electrophoresed on a 6% denaturing PAGE gel after RNase A and T1 digestion. Lane 1 contains 2 μg of total RNA from HepG2 cells transfected with pHBV; lane 2 contains 2 μg of total RNA from HepG2 cells transfected with pHBV and pHBV DN; lane 3 contains 2 μg of total RNA from HepG2 cells transfected with pHBV DN alone (the BamHI-EcoRI fragment is missing in this construct). The remaining lanes show RNA that was extracted from HepG2-derived core particles and then probed as in lanes 1-3. Each lane was loaded with half of the core associated RNA extracted from a 10 cm dish. Lane 4 contains core particle associated RNA from cells transfected with pHBV. Lane 5 contains core particle associated RNA from cells transfected with pHBV and pHBV DN. Lane 6 contains core particle associated RNA from cells transfected with pHBV DN alone. There was a 90% reduction in encapsulation of “wild type” pregenomic RNA when pHBV DN was co-transfected with the wild type HBV DNA expressing plasmid, whereas no significant reduction in viral RNA was observed in experiments performed with total cellular RNA. The riboprobe used in this experiment protects pregenomic and pre-S1 transcripts, both of which were absent in the pHBV DN transfected cells. “Wild type” pregenomic viral RNA is incapable of being encapsulated in the presence of mutant core protein because of inefficient core particle assembly. Cell lysates derived from HepG2 cells previously transfected with pHBV alone, pHBV and pHBV DN together, and pHBV DN alone were sedimented on a 20% w/v sucrose cushion for one hour at 500,000 g. Under these experimental conditions non-particulate core protein and HBeAg were left in solution (Zhou et al., supra). The pellet was analyzed for core protein by 12.5% SDS-PAGE electrophoresis, and analyzed on a Western blot using polyclonal anti-HBc antibodies as probes. The viral core particles were derived from: lane 1, cells transfected with pHBV; lane 2, cells transfected with pHBV and pHBV DN; lane 3, cells transfected with pHBV DN alone; lane 4, HepG2 2215 cells (positive control). Lane 5 contains 100 μg of cell lysate in RIPA buffer not subjected to ultracentrifugation and extracted from HepG2 2215 cells to show enrichment of core particles by the pelleting technique (positive control). The protein in lane 6 was derived from the pellets of untransfected HepG2 cells (negative control). A protein band of 21.5 kd, corresponding to the intact “wild type” HBV core protein, was detected only in the pellet derived from HepG2 cells transfected with pHBV. In the pellet of cells transfected with the pHBV DN plasmid, an immunoreactive core protein band of 11.5 kd was detected. This protein was substantially smaller than the predicted size of the full length core-surface fusion protein derived from the pHBV DN (about 38 kd).

To determine whether the HBV core dominant negative mutant HBV DN can make hepatoma cell lines resistant to HBV replication, the HBV DN coding sequence was cloned into the retroviral vector pBabe Puro (pBP), which contains a puromycin selectable marker. The resulting vector is named pBPHBV DN. Recombinant retroviral stocks were obtained after transfecting pBPHBV DN into the packaging cell line PA317. The stocks were then used to infect HepG2 and HepG2 2215 cell lines. The HepG2 2215 cells constitutively produce wild type HBV virions due to the stable integration of a head to tail dimer of HBV. Pools of stably transduced clones were grown in the presence of puromycin. HBV DNA was purified from the core particles and analyzed by Southern blot. HepG2 2215 transduced by the pBP HBV DN vector showed a 90% reduction in HBV replication when compared to HepG2 2215 cells transduced by the pBP vector. This result demonstrates a striking reduction of HBV replicative intermediates in core particles, even in a cell line that constitutively expresses all the viral gene products and replicative forms of the virus.

The Flag tagged dominant negative form of the HBV DN sequence was also cloned into the adenoviral vector pAdBglII to generate the vector pAdHBV DN. This vector contains a multiple cloning site flanked by the CMV EI promoter and by adenovirus 5 sequences. The adenovirus 5 sequences allow homologous recombination and reconstitution of a recombinant replication incompetent adenovirus after cotransfection in 293 cells (Graham et al., the Human press, Vol 7, 109-128, 1991). The plasmid pAdHBV DN was then introduced, along with pHBV, into HCC cells by transient transfection, inhibiting HBV replication by 80%. Adenoviral vectors such as pAd HBV DN can be used to generate a replication incompetent adenovirus by homologous recombination, and can express the HBV DN polypeptide in the liver.

Inhibition of DHBV replication Substantial suppression of DHBV replication was obtained by co-transfecting pCMV DHBV with the plasmid pBK. The pBK plasmid encodes a DHBV core protein which lacks the last carboxyterminal five amino acids, fused to a surface protein which lacks the aminoterminal first four amino acids. When shorter core fragments were fused in frame to a surface protein lacking the aminoterminal first four amino acids (plasmid pSK), to the Pol gene product (pNX), or to the pre-S gene product (pSK), there was little or no effect on DHBV replication. This result indicated that both the core protein and the surface protein extension were important for exerting an inhibitory effect on “wild type” DHBV replication, presumably by disrupting nucleocapsid assembly. The core portion of the chimeric mutant polypeptide interacts with the wild type core protein, preventing formation of intact nucleocapsids and thus encapsulation of the DHBV pregenome. A construct that expressed only the DHBV core protein (pK) was incapable of inhibiting DHBV replication, while a plasmid that expressed the same core portion as the pBK plasmid but fused to the polymerase gene was incapable of inhibiting “wild type” DHBV replication. Included in this analysis was a Southern Blot analysis of cytosolic derived nucleocapsid DNA from transfected LMC cells, hybridized to a full length DHBV DNA probe. LMC cells were transfected with 10 μg of pCMV DHBV together with 10 μg of mutant plasmids pSK (lane 2), pBK (lane 3), pSK (lane 4, the same as lane 2), pK (lane 5), or pNX (lane 6). The last lane contains the cytosolic derived nucleocapsid DNA from a LMC cell line stably transfected with a head-to-tail DHBV dimer as a positive control. Replication of “wild type” DHBV was inhibited by the dominant negative core mutant construct BK. Therapeutic Use The mutant polypeptides of the invention can be provided exogenously to a target cell of an animal suspected of harboring a hepadnavirus infection by any appropriate method, for example by oral, parenteral, transdermal, or transmucosal administration. The mutant polypeptide can be administered in a sustained release formulation using a biodegradable biocompatible polymer, or by on-site delivery using micelles, gels or liposomes. Therapeutic doses can be, but are not necessarily, in the range of 0.01-100.0 mg/kg body weight, or a range that is clinically determined to be appropriate by those skilled in the art.

The polypeptides useful in a method of the invention, or as candidate agents in a method of the invention, can be purified using conventional methods of protein isolation known to one skilled in the art. These methods include, but are not limited to, precipitation, chromatography, immunoadsorption, or affinity techniques (see, e.g., Scopes, R. Protein Purification: Principles and Practice, 1982 Springer Verlag, N.Y.). The polypeptide can be purified from starting material that is derived from a genetically engineered cell line. One useful method of purification involves expressing the polypeptide as a fusion protein encoded by a glutathione-S-transferase vector, purifying the resulting fusion protein by GST-GSH affinity chromatography, and removing the GST portion of the fusion polypeptide by thrombin cleavage. Alternatively, a synthetic mutant polypeptide can be prepared by automated peptide synthesis (see, e.g., Ausubel et al., eds. Current Protocols in Molecular Biology, John Wiley & Sons, publ. N.Y. 1987, 1989; Sambrook et al. (1989), Molecular Cloning: A Laboratory Manual (2d ed.), CSH Press).

Therapeutic administration of a mutant polypeptide can also be accomplished using gene therapy techniques. A nucleic acid that included a promoter operatively linked to a sequence encoding a polypeptide of the invention is used to generate high-level expression of the polypeptide in cells transfected with the nucleic acid. Gene transfer can be performed ex vivo or in vivo. To administer the nucleic acid ex vivo, cells can be removed from the body of the patient, transfected with the nucleic acid encoding the mutant polypeptide, and returned to the patient's body. Alternatively the nucleic acid can be administered in vivo, by transfecting the nucleic acid into target cells (e.g., hepatocytes) so that the mutant polypeptide is expressed in situ.

The nucleic acid molecule is contained within a non-replicating linear or circular DNA or RNA molecule, or within an autonomously replicating plasmid or viral vector, or may be integrated into the host genome. Any vector that can transfect a cell can be used in the methods of the invention. Preferred vectors are viral vectors, including those derived from replication-defective hepatitis virus (e.g., HBV and HCV), retrovirus (see, e.g., WO89/07136; Rosenberg et al., N. Eng. J. Med. 323(9):570-578, 1990; Miller et al., 1993 supra), adenovirus (see, e.g., Morsey et al., J. Cell. Biochem., Supp. 17E, 1993; Graham et al., in Murray, ed., Methods in Molecular Biology: Gene Transfer and Expression Protocols. Vol. 7, Clifton, N.J.: the Human Press 1991:109-128), adeno-associated virus (Kotin et al., Proc. Natl. Acad. Sci. USA 87:2211-2215, 1990), replication defective herpes simplex virus (HSV; Lu et al., Abstract, page 66, Abstracts of the Meeting on Gene Therapy, Sep. 22-26, 1992, Cold Spring Harbor Laboratory, Cold Spring Harbor, N.Y.), and any modified versions of these vectors. Other preferred viral vectors include those modified to target a specific cell type (see, e.g., Kan et al. WO 93/25234; Kasahara et al. Science, 266:1373-76, 1994; Dornburg et al. WO 94/12626; Russell et al. WO 94/06920). Methods for constructing expression vectors are well known in the art (see, e.g., Molecular Cloning: A Laboratory Manual, Sambrook et al., eds., Cold Spring Harbor Laboratory, 2nd Edition, Cold Spring Harbor, N.Y., 1989).

Appropriate regulatory sequences can be inserted into the vectors of the invention using methods known to those skilled in the art, e.g., by homologous recombination (Graham et al., J. Gen. Virol. 36:59-72, 1977), or by other appropriate methods (Sambrook et al., eds., supra). Promoters are inserted into the vectors so that they are operatively linked 5′ to the nucleic acid sequence encoding the mutant polypeptide. Any promoter that is able to initiate transcription in a target cell can be used in the invention. For example, non-tissue specific promoters, such as the cytomegalovirus (DeBernardi et al., Proc. Natl. Acad. Sci. USA 88:9257-9261, 1991, and references therein), mouse metallothionine I gene (Hammer, et al., J. Mol. Appl. Gen. 1:273-288, 1982), HSV thymidine kinase (McKnight, Cell, 31:355-365, 1982), and SV40 early (Benoist et al., Nature, 290:304-310, 1981) promoters may be used. Preferred promoters for use in the invention are hepatocyte-specific promoters, the use of which ensures that the mutant polypeptides are expressed primarily in hepatocytes. Preferred hepatocyte-specific promoters include, but are not limited to the albumin, alpha-fetoprotein, alpha-1-antitrypsin, retinol-binding protein, and asialoglycoprotein receptor promoters. Additional viral promoters and enhancers, such as those from herpes simplex virus (types I and II), hepatitis virus (Types A, B, and C), and Rous sarcoma virus (RSV; Fang et al., Hepatology 10:781-787, 1989), can also be used in the invention.

The mutant polypeptides of the invention, and the recombinant vectors containing nucleic acid sequences encoding them, may be used in therapeutic compositions for preventing or treating HBV infection. The therapeutic compositions of the invention may be used alone or in admixture, or in chemical combination, with one or more materials, including other mutant polypeptides or recombinant vectors, materials that increase the biological stability of the oligonucleotides or the recombinant vectors, or materials that increase the ability of the therapeutic compositions to penetrate hepatocytes selectively. The therapeutic compositions of the invention can be administered in pharmaceutically acceptable carriers (e.g., physiological saline), which are selected on the basis of the mode and route of administration, and standard pharmaceutical practice. Suitable pharmaceutical carriers, as well as pharmaceutical necessities for use in pharmaceutical formulations, are described in Remington's Pharmaceutical Sciences, a standard reference text in this field.

The therapeutic compositions of the invention can be administered in dosages determined to be appropriate by one skilled in the art. An appropriate dosage is one which effects a reduction in a disease caused by HBV infection. It is expected that the dosages will vary, depending upon the pharmacokinetic and pharmacodynamic characteristics of the particular agent, and its mode and route of administration, as well as the age, weight, and health (including renal and hepatic function) of the recipient; the nature and extent of the disease; the frequency and duration of the treatment; the type of, if any, concurrent therapy; and the desired effect. It is expected that a useful dosage contains between about 0.1 to 100 mg of active ingredient per kilogram of body weight. Ordinarily a dosage of 0.5 to 50 mg, and preferably, 1 to 10 mg of active ingredient per kilogram of body weight per day given in divided doses, or in sustained release form, is appropriate.

The therapeutic compositions of the invention may be administered to a patient by any appropriate mode, e.g., parenterally, as determined by one skilled in the art. Alternatively, it may by necessary to administer the treatment surgically to the target tissue. The treatments of the invention may be repeated as needed, as determined by one skilled in the art.

The invention also includes any other methods which accomplish in vivo transfer of nucleic acids into target cells. For example, the nucleic acids may be packaged into liposomes, non-viral nucleic acid-based vectors, erythrocyte ghosts, or microspheres (microparticles; see, e.g., U.S. Pat. No. 4,789,734; U.S. Pat. No. 4,925,673; U.S. Pat. No. 3,625,214; Gregoriadis, Drug Carriers in Biology and Medicine, pp. 287-341 (Academic Press, 1979)). Further, delivery of mutant polypeptides be accomplished by direct injection of their nucleic acid coding sequences into target tissues, for example, in a calcium phosphate precipitate or coupled with lipids, or as “naked DNA”.

Mutant core polypeptides and core-surface fusion proteins of the invention can be tested for their ability to inhibit hepadnavirus replication in an animal model. For example, candidate polypeptides can be injected into an animal that is infected with a hepadnavirus, e.g., a woodchuck, duck, or ground squirrel harboring its respective hepatitis B virus variants (see, e.g., Mason et al., J. Virol. 36:829, 1980; Schodel et al., in Molecular Biology of hepatitis B virus, CRC press, Boca Raton, p. 53-80, 1991; Summers et al., Proc. Natl. Acad. Sci. USA, 75:4533-4537, 1978). Candidate polypeptides can also be analyzed in transgenic animal strains developed for the purpose of studying hepadnaviral gene expression (see, e.g., Babinet et al., Science, 230:1160-63, 1985; Burk et al., J. Virol. 62:649-54, 1988; Chisari et al., Science 230:1157-60, 1985; Chisari, in Current Topics in Microbiology and Immunology, p. 85-101, 1991). Candidate polypeptides of the invention can also be tested in animals that are naturally infected with HBV, e.g., in chimpanzees, by administering the polypeptides, or the nucleic acids encoding them, to the animal by one of the methods discussed above, or by other standard methods.

Other Embodiments

From the above description, one skilled in the art can easily ascertain the essential characteristics of the present invention, and without departing from the spirit and scope thereof, can make various changes and modifications of the invention to adapt it to various usages and conditions.

All publications cited herein are fully incorporated by reference in their entirety.

Other embodiments are within the claims set forth below.

What is claimed is:

1. A nucleic acid encoding a polypeptide that
(a) comprises a first amino acid sequence of at least 70 amino acids in length that is identical to a region of a wild type HBV core protein; and
(b) lacks a second amino acid sequence of the wild type HBV core protein, wherein the second sequence comprises the carboxyterminal three amino acids of the wild type HBV core protein and does not exceed nine amino acids in length.

2. The nucleic acid of claim 1, wherein the carboxyterminal amino acid of the first amino acid sequence is selected from the group consisting of each of the amino acids between position 174 and position 180 of SEQ ID NO: 12, inclusive.

3. A nucleic acid encoding a polypeptide that
(a) comprises a first amino acid sequence of at least 70 amino acids in length that is identical to a region of a wild type hepadnavirus core protein;
(b) lacks a second amino acid sequence of the wild type hepadnavirus core protein, wherein the second sequence comprises the carboxyterminal three amino acids of the wild type hepadnavirus core protein; and
(c) comprises a third amino acid sequence that is identical to a portion of a wild type hepadnavirus surface protein.

4. The nucleic acid of claim 3, wherein the second amino acid sequence does not exceed 100 amino acids in length.

5. The nucleic acid of claim 3, wherein the carboxyterminal amino acid of the first amino acid sequence corresponds to a position selected from the group consisting of each of amino acids 71 to 180 of SEQ ID NO: 12, inclusive.

6. A nucleic acid encoding a polypeptide comprising
(a) a first amino acid sequence of at least 70 amino acids in length that is identical to a region of a wild type hepadnavirus core protein; and
(b) a second amino acid sequence that is identical to a portion of a wild type hepadnavirus surface protein.

7. A vector comprising the nucleic acid of claim 1.

8. A vector comprising the nucleic acid of claim 2.

9. A vector comprising the nucleic acid of claim 3.

10. A vector comprising the nucleic acid of claim 4.

11. A vector comprising the nucleic acid of claim 5.

12. A vector comprising the nucleic acid of claim 6.

13. A cultured host cell comprising the nucleic acid of claim 1.

14. A cultured host cell comprising the nucleic acid of claim 2.

15. A cultured host cell comprising the nucleic acid of claim 3.

16. A cultured host cell comprising the nucleic acid of claim 4.

17. A cultured host cell comprising the nucleic acid of claim 5.

18. A cultured host cell comprising the nucleic acid of claim 6.
